(19) United States
(12) Patent Application Publication
(10) Pub. No.: US 2024/0319171 A1
(43) Pub. Date: Sep. 26, 2024

(54) METHOD FOR SCREENING SUBSTANCE, SYSTEM FOR SCREENING SUBSTANCE, METHOD FOR PRODUCING A CLUMP FROM STEM CELLS, AND METHOD FOR PRODUCING DIFFERENTIATED CELLS

(71) Applicants: Koji TANABE, Palo Alto, CA (US); I Peace, Inc., Palo Alto, CA (US)

(72) Inventors: Koji TANABE, Palo Alto, CA (US); Sakura TAMAKI, Kyoto-shi (JP)

(73) Assignees: Koji TANABE, Palo Alto, CA (US); I Peace, Inc., Palo Alto, CA (US)

(21) Appl. No.: 18/615,184

(22) Filed: Mar. 25, 2024

Related U.S. Application Data
(60) Provisional application No. 63/491,876, filed on Mar. 23, 2023.

(51) Int. Cl. G01N 33/50; C12N 5/077

(52) U.S. Cl. CPC G01N 33/5044; C12N 5/0656; C12N 2503/00; C12N 2506/09

(57) ABSTRACT

According to the present disclosure, there is provided a method for screening a substance, which includes inducing pluripotent stem cells from somatic cells of a subject; inducing differentiated cells from the pluripotent stem cells; adding a substance to the differentiated cells and culturing the differentiated cells; and determining whether or not to administering or applying the substance to the subject based on a response of the differentiated cells to the substance.

### BACKGROUND

### Field

The present invention relates to cell technology and relates to a method for screening a substance, a system for screening a substance, a method for producing a clamp from stem cells, and a method for producing differentiated cells.

### Description of Related Art

Induced pluripotent stem (iPS) cells are cells that have two characteristic abilities. One is the ability to transform to any cell that forms the body. The other is to have a semi-permanent proliferation ability. Due to having these two abilities, iPS cells are capable of being applied to a transplantation treatment having no rejection reaction by preparing iPS cells from autologous somatic cells and allowing the prepared iPS cells to transform to somatic cells of interest. Therefore, iPS cells are considered to be a promising technology in regenerative medicine (see, for example, patent literatures 1 to 4 as well as non patent literatures 1 and 2).

### Prior Art Document

### Patent Literature

Patent Literature 1: International Publication No. 2000/70070

Patent Literature 2: International Publication No. 2010/008054

Patent Literature 3: International Publication No. 2012/029770

Patent Literature 4: International Publication No. 2015/046229

### Non Patent Literature

Non Patent Literature 1: Nature 448, 313-317

Non Patent Literature 2: Nature Biotechnol 26(3): 313-315, 2008

### SUMMARY

An object of the present invention is to provide a method for screening a substance using stem cells, a system for screening a substance, a method for producing a clamp from stem cells, and a method for producing differentiated cells from stem cells.

A method for screening a substance according to an embodiment of the present invention includes inducing pluripotent stem cells from somatic cells of a subject; inducing differentiated cells from the pluripotent stem cells; adding a substance to the differentiated cells and culturing the differentiated cells; and determining whether or not to administering or applying the substance to the subject based on a response of the differentiated cells to the substance.

In the above-described method for screening a substance, the subject from which the somatic cells are provided and the subject to which the substance is administered or applied may be the same person.

In the above-described method for screening a substance, the substance may be an additive of a beauty product, a cosmetic product, a hair tonic, or a hair growth promoter.

In the above-described method for screening a substance, the substance may be an additive of a pharmaceutical drug.

In the above-described method for screening a substance, the substance may be a vitamin.

In the above-described method for screening a substance, the substance may be at least one of ascorbic acid and retinol.

In the above-described method for screening a substance, the substance may be an antiseptic agent.

In the above-described method for screening a substance, the differentiated cells may be skin cells.

In the above-described method for screening a substance, the response of the differentiated cells may be production of a product by the differentiated cells.

In the above-described method for screening a substance, the product may be collagen.

In the above-described method for screening a substance, the response of the differentiated cells may be a survival rate of the differentiated cells.

A method for administering or applying a substance according to an embodiment of the present invention includes inducing pluripotent stem cells from somatic cells of a subject; inducing differentiated cells from the pluripotent stem cells; adding a substance to the differentiated cells and culturing the differentiated cells; determining whether or not to administering or applying the substance to the subject based on a response of the differentiated cells to the substance; and administering or applying the substance to the subject, where the substance has been determined to be administered or applied.

In the above-described method for administering or applying a substance, the subject from which the somatic cells are provided and the subject to which the substance is administered or applied may be the same person.

In the above-described method for administering or applying a substance, the substance may be an additive of a beauty product, a cosmetic product, a hair tonic, or a hair growth promoter.

In the above-described method for administering or applying a substance, the substance may be an additive of a pharmaceutical drug.

In the above-described method for administering or applying a substance, the substance may be a vitamin.

In the above-described method for administering or applying a substance, the substance may be at least one of ascorbic acid and retinol.

In the above-described method for administering or applying a substance, the substance may be an antiseptic agent.

In the above-described method for administering or applying a substance, the differentiated cells may be skin cells.

In the above-described method for administering or applying a substance, the response of the differentiated cells may be production of a product by the differentiated cells.

In the above-described method for administering or applying a substance, the product may be collagen.

In the above-described method for administering or applying a substance, the response of the differentiated cells may be a survival rate of the differentiated cells.

A system for screening a substance according to an embodiment of the present invention includes a data acquirer that is configured to acquire data on a response of differentiated cells in a case where a substance is added to the differentiated cells induced from pluripotent stem cells induced from somatic cells of a subject and the differentiated cells are cultured; an evaluator that is configured to evaluate the substance based on the data on the response of the differentiated cells; and a determination unit that is configured to determine whether or not to administer or apply the substance to the subject based on the evaluation.

In the above-described system for screening a substance, the subject from which the somatic cells are provided and the subject to which the substance is administered or applied may be the same person.

In the above-described system for screening a substance, the substance may be an additive of a beauty product, a cosmetic product, a hair tonic, or a hair growth promoter.

In the above-described system for screening a substance, the substance may be an additive of a pharmaceutical drug.

In the above-described system for screening a substance, the substance may be a vitamin.

In the above-described system for screening a substance, the substance may be at least one of ascorbic acid and retinol.

In the above-described system for screening a substance, the substance may be an antiseptic agent.

In the above-described system for screening a substance, the differentiated cells may be skin cells.

In the above-described system for screening a substance, the response of the differentiated cells may be production of a product by the differentiated cells.

In the above-described system for screening a substance, the product may be collagen.

In the above-described system for screening a substance, the response of the differentiated cells may be a survival rate of the differentiated cells.

A method for producing a clamp from stem cells according to an embodiment of the present invention includes seeding stem cells as single cells in a culture vessel having a low adhesive surface; and subjecting the stem cells to three-dimensional culture in a culture medium containing an extracellular substrate in the culture vessel to form a clamp from the stem cells.

In the above-described method for producing a clamp from stem cells, the stem cells may be iPS cells.

In the above-described method for producing a clamp from stem cells, the extracellular substrate may include laminin.

In the above-described method for producing a clamp from stem cells, the surface of the culture vessel may have low adhesiveness.

A method for producing differentiated cells according to the embodiment of the present invention includes seeding stem cells in a first culture vessel having a low adhesive surface; subjecting the stem cells to three-dimensional culture in a culture medium in the first culture vessel to form a clamp from the stem cells; and seeding the clamp in a second culture vessel and inducing differentiated cells from the clamp.

In the above-described method for producing differentiated cells, in the seeding, the stem cells may be seeded as single cells.

In the above-described method for producing differentiated cells, the stem cells may be iPS cells.

In the above-described method for producing differentiated cells, the iPS cells may be derived from skin cells.

In the above-described method for producing differentiated cells, the culture medium includes an extracellular substrate. The extracellular substrate may include laminin.

In the above-described method for producing differentiated cells, the surface of the first culture vessel has low adhesiveness.

In the above-described method for producing differentiated cells, the surface of the second culture vessel may be coated with gelatin.

In the above-described method for producing differentiated cells, the differentiated cells may be skin cells.

In the above-described method for producing differentiated cells, the skin cells may be dermal cells or epidermal cells.

A method for producing a clamp from stem cells according to an embodiment of the present invention includes seeding stem cells in a stirring culture vessel; stirring a culture medium in the stirring culture vessel at a speed at which the stem cells do not adhere to a bottom surface of the stirring culture vessel and at least 20% or more of the stem cells come into contact with the bottom surface of the stirring culture vessel; and forming a clamp from the stem cells in the stirring culture vessel.

In the above-described method for producing a clamp from stem cells, the stem cells may be iPS cells.

In the above-described method for producing a clamp from stem cells, the stem cells may be frozen in the seeding the stem cells in the stirring culture vessel.

In the above-described method for producing a clamp from stem cells, the stem cells may be seeded in the stirring culture vessel together with a freezing solution in the seeding the stem cells in the stirring culture vessel.

The above-described method for producing a clamp from stem cells may further include differentiating the clamp into at least one of the endotherm, the mesoderm, and the ectoderm.

A method for producing differentiated cells according to the embodiment of the present invention may include seeding stem cells in a stirring culture vessel; stirring a culture medium in the stirring culture vessel at a speed at which the stem cells do not adhere to a bottom surface of the stirring culture vessel and at least 20% or more of the stem cells come into contact with the bottom surface of the stirring culture vessel; forming a clamp from the stem cells in the stirring culture vessel; and seeding the clamp in a culture vessel and inducing differentiated cells from the clamp by two-dimensional culture.

In the above-described method for producing differentiated cells, the stem cells may be iPS cells.

In the above-described method for producing differentiated cells, the iPS cells may be derived from skin cells.

In the above-described method for producing differentiated cells, the differentiated cells may be skin cells.

In the above-described method for producing differentiated cells, the skin cells may be dermal cells or epidermal cells.

In the above-described method for producing differentiated cells, the stem cells may be frozen in the seeding the stem cells in the stirring culture vessel.

In the above-described method for producing differentiated cells, the stem cells may be seeded in the stirring culture vessel together with a freezing solution in the seeding the stem cells in the stirring culture vessel.

According to the present invention, it is possible to provide a method for screening a substance using stem cells, a system for screening a substance, a method for producing a clamp from stem cells, and a method for producing differentiated cells from stem cells.

### BRIEF DESCRIPTION OF DRAWINGS

FIG. 1 is a view schematically illustrating a system for screening a substance.

FIGS. 2A and 2B show photographic images of clamps according to Examples.

FIG. 3 shows a photographic image of clamps according to Examples.

FIGS. 4A to 4C show photographic images of cells according to Examples.

FIG. 5 shows dot plots from flow cytometry according to Examples.

FIG. 6 shows a photographic image of cells according to Comparative Examples.

FIGS. 7A to 7C show photographic images of clamps according to Examples.

FIG. 8 shows a dot plot from flow cytometry according to Examples.

FIGS. 9A to 9C show graphs of an expression level of a marker of the endotherm, an expression level of a marker of the mesoderm, and an expression level of a marker of the ectoderm according to Examples.

FIGS. 10A and 10B show photographic images of cells according to Examples.

FIGS. 11A to 11D show photographic images of cells according to Examples.

FIG. 12 shows dot plots from flow cytometry according to Examples.

FIG. 13 shows a photographic image of cells according to Comparative Examples.

FIGS. 14A to 14B show graphs of the production amount of collagen according to Examples.

FIGS. 15A and 15B show graphs of cell survival rates according to Examples.

### DETAILED DESCRIPTION

Hereinafter, embodiments of the present invention will be described in detail. It is noted that the following embodiments show examples of devices and methods for embodying the technical idea of the present invention, and the technical idea of the present invention does not specify the combination of constituent members and the like to those described below. The technical idea of the present invention is capable of being modified in various ways within the scope of the patent claims.

### First Embodiment of Method for Producing Clamp From Stem Cells

A first embodiment of a method for producing a clamp from stem cells includes seeding stem cells in a culture vessel having a low adhesive surface; and subjecting the stem cells to three-dimensional culture (suspension culture) in a culture medium containing an extracellular substrate in the culture vessel having a low adhesive surface, to form a clamp from the stem cells. The clamp is also referred to as an embryoid body (EB). The clamp is, for example, positive for a marker of the endotherm. The clamp is, for example, positive for a marker of the mesoderm. The clamp is, for example, positive for a marker of the ectoderm. Examples of the marker of the endotherm include SOX17. Examples of the marker of the mesoderm include Brachyury (T). Examples of the marker of the ectoderm include PAX6.

The stem cell is, for example, a pluripotent stem cell. The pluripotent stem cell is, for example, an induced pluripotent stem cell (iPS cell) or an embryonic stem cell (ES cell). The stem cell may be derived from a human or may be derived from a non-human animal. The iPS cell may be derived from, for example, a skin cell, a blood cell, a dental pulp stem cell, a keratinocyte, a hair papilla cell, an oral epithelial cell, a somatic stem/progenitor cell, or a urinary bladder epithelial cell.

The skin cell may be a dermal cell such as a fibroblast and an epidermal cell such as a keratinocyte. The skin cell may be a fibroblast. The blood cell is a nucleated cell such as a monocyte, a neutrophil, a macrophage, an eosinophil, a basophil, or a lymphocyte, and does not include an erythrocyte, a granulocyte, and a thrombocyte. The blood cell may be, for example, an endothelial progenitor cell, a blood stem/progenitor cell, a T cell, or a B cell. The T cell is, for example, an αβT cell.

In seeding stem cells in the culture vessel having the low adhesive surface, the stem cells may be seeded as single cells in the culture vessel. The stem cells are dissociated into single cells, for example, by being suspended in a solution containing a cell dissociating agent. In a case where the stem cells are seeded as single cells in the culture vessel, it is possible to suppress the mutual aggregation of the clamps in a case where the stem cells form the clamps. The cell dissociating agent is, for example, a cell dissociating enzyme. Examples of the cell dissociating agent include TrypLE (registered trademark, Thermo Fisher Scientific, Inc.) and trypsin. It is noted that a method for dissociating stem cells into single cells is not limited to the method for using a cell dissociating enzyme. For example, the stem cells may be dissociated into single cells by using, for example, 0.1 to 10 mM ethylenediaminetetraacetic acid (EDTA) as a cell dissociating agent. In a case where a cell dissociating agent that is not an enzyme is used, the cell survival rate and the ability to form a clamp may be improved. The stem cells are suspended as single cells in a culture medium containing, for example, an extracellular substrate, and then seeded in the culture vessel. However, the stem cells may be seeded in the culture vessel without being dissociated into single cells.

The culture vessel having the low adhesive surface has low adhesiveness to cells, proteins, peptides, and compounds having a low molecular weight. The culture vessel having the low adhesive surface is, for example, a culture vessel having a hydrophilic surface. The culture vessel having a hydrophilic surface is produced, for example, by coating the surface of a culture vessel with a hydrophilic polymer. The hydrophilic polymer may be bonded to the surface of the culture vessel by crosslinking. In a case where the stem cells are seeded as single cells in the culture vessel having the hydrophilic surface, it is possible to subject the stem cells to three-dimensional culture without the stem cells adhering to the surface of the cultured solution. Examples of the culture vessel having the hydrophilic surface include PrimeSurface (registered trademark, Sumitomo Bakelite Co., Ltd.).

As the culture medium, for example, a stem cell culture medium such as a human ES/iPS medium such as Primate ES Cell Medium (REPROCELL Incorporated) is capable of being used.

However, the stem cell culture medium is not limited to this, and various stem cell culture media are capable of being used. For example, the following culture medium may be used: Primate ES Cell Medium, Reprostem, ReproFF, ReproFF2, ReproXF (REPROCELL Incorporated), mTeSR1, TeSR2, TeSRE8, ReproTeSR (STEMCELL Technologies Inc.) PluriSTEM (registered trademark) Human ES/iPS Medium (Merck KGaA), NutriStem (registered trademark) XF/FF Culture Medium for Human iPS and ES Cells, Pluriton reprogramming medium (Stemgent), PluriSTEM (registered trademark), Stemfit AK02N, Stemfit AK03 (Ajinomoto Co., Inc.), ESC-Sure (registered trademark) serum and feeder free medium for hESC/iPS (Applied StemCell), L7 (registered trademark) hPSC Culture System (LONZA), PluriQ (MTI-GlobalStem), and the like.

Examples of the extracellular substrate include laminin, collagen, cadherin, and fibronectin, as well as vitronectin, PEG, and PVA. The extracellular substrate may be contained in Matrigel (registered trademark, Corning Inc.). By adding the extracellular substrate to the culture medium, the stem cells efficiently form the clamps.

### Second Embodiment of Method for Producing Clamp From Stem Cells

A second embodiment of a method for producing a clamp from stem cells includes seeding stem cells in a stirring culture vessel; stirring a culture medium in the stirring culture vessel at a speed at which the stem cells do not adhere to a bottom surface of the stirring culture vessel and at least 20% or more of the stem cells come into contact with the bottom surface of the stirring culture vessel; and forming a clamp from the stem cells in the stirring culture vessel. The clamp is, for example, positive for a marker of the endotherm. The clamp is, for example, positive for a marker of the mesoderm. The clamp is, for example, positive for a marker of the ectoderm.

The stem cells are the same as those in the first embodiment. In addition, in the second embodiment, the frozen stem cells may be seeded in the stirring culture vessel together with the freezing solution.

The stirring culture vessel includes a container for internally housing a culture medium and a stirring device for stirring a culture medium in a container. A bioreactor may be used as the stirring culture vessel. Examples of the bioreactor include ABLE (registered trademark, REPROCELL Incorporated). In the second embodiment, the culture medium that is supplied into the stirring culture vessel is the same as the culture medium in the first embodiment. However, in the second embodiment, the culture medium may contain an extracellular substrate, or the culture medium may not contain an extracellular substrate.

In the stirring culture vessel, the stem cells are cultured so that the stem cells in the container do not adhere to the inner surface of the container by stirring the culture medium in the container with the stirring device. Here, in a case where the stirring speed for the culture medium is fast, the stem cells float in the container, and in a case where the stirring speed for the culture medium is slow, the stem cells precipitate in the container.

In the stirring culture vessel, the culture medium in the container is stirred at a speed at which the stem cells do not adhere to the bottom surface inside the container and, among the stem cells, at least 20% or more and less than 100%, 20% or more and 95% or less, 20% or more and 90% or less, 20% or more and 80% or less, 30% or more and less than 100%, 30% or more and 95% or less, 30% or more and 90% or less, 30% or more and 80% or less, 50% or more and less than 100%, 50% or more and 95% or less, 50% or more and 90% or less, 50% or more and 80% or less, 80% or more and less than 100%, 80% or more and 95% or less, 80% or more and 90% or less, 85% or more and less than 100%, 85% or more and 95% or less, 85% or more and 90% or less, 90% or more and less than 100%, 90% or more and 95% or less, or 95% or more and less than 100% come into contact with the bottom surface inside the container. Here, the contact refers to that stem cells come into contact with the bottom surface to be movable by stirring the culture medium. The stem cells may move on the bottom surface while being in contact with the bottom surface inside the container. In a case where the culture medium is stirred at a speed at which at least 20% or more of the stem cells come into contact with the bottom surface of the container, the stem cells efficiently form clamps.

After the clamps are formed, the stirring speed may be increased to a speed at which less than 20% of the clamps come into contact with the bottom surface inside the container. In a case where the stirring speed is increased after the clamps are formed, the sizes of the plurality of clamps become uniform, and the efficiency of differentiation into the three germ layers tends to be improved.

### Method for Producing Differentiated Cells

The embodiment of the method for producing differentiated cells includes seeding, in a culture vessel, the clamp produced in the first or second embodiment of the method for producing the clamp from the stem cells and inducing differentiated cells from the clamp. One or more, two or more, or five or more clamps may be seeded in the culture vessel per 1 cm². In a case where the differentiated cells are induced from the clamp, the cells may be subjected to two-dimensional culture or may be subjected to three-dimensional culture. The differentiated cell is, for example, a skin cell. In the case of producing skin cells, the iPS cells that form a clamp are preferably derived from skin cells. The skin cell may be a dermal cell or an epidermal cell. The skin cell may be a fibroblast. The inner surface of the culture vessel in which the clamp is seeded may be coated with gelatin, laminin, collagen, cadherin, fibronectin, or vitronectin. The culture vessel may be a dish. Examples of the culture medium for inducing skin cells from the clamp include, but are not particularly limited to, a Dulbecco's modified Eagle medium (DMEM) to which fetal bovine serum (FBS) has been added.

At the time when the cells have become confluent in the culture vessel, the cells may be detached from the culture vessel with a stripping agent such as trypsin or the like to make the cells single cells, which are subsequently subcultured. The passage may be repeated. Then, using a fibroblast marker such as CD13, it may be confirmed that the cells have differentiated into fibroblasts. In addition, using undifferentiation markers such as TRA1-60, SSEA-4, and SSEA-5, it may be confirmed whether or not undifferentiated iPS cells remain in the cells.

### Method for Screening Substance

A method for screening a substance includes inducing pluripotent stem cells from somatic cells of a subject; inducing differentiated cells from the pluripotent stem cells; adding a substance to the differentiated cells and culturing the differentiated cells; and determining whether or not to administer or apply the substance to the subject based on a response of the differentiated cells to the substance.

The subject is a human. The somatic cells of the subject are acquired from the subject in advance. The somatic cell is not particularly limited; however, it is, for example, a skin cell, a blood cell, a dental pulp stem cell, a keratinocyte, a hair papilla cell, an oral epithelial cell, a somatic stem/progenitor cell, or a urinary bladder epithelial cell. The somatic cell is preferably a skin cell. Inducing pluripotent stem cells from the somatic cells of the subject is carried out in vitro.

A method for inducing pluripotent stem cells from the somatic cells is not particularly limited. For example, iPS cells are induced from somatic cells by introducing a reprogramming factor into the somatic cells.

The reprograming factor to be introduced into somatic cells is, for example, RNA. The RNA is, for example, an mRNA. The reprograming factor to be introduced into cells includes, for example, OCT such as OCT3/4, SOX such as SOX2, KLF such as KLF4, and MYC such as c-MYC. M₃O, which is obtained by improving OCT3/4, may be used as a reprograming factor. In addition, the reprograming factor may include at least one factor selected from the group consisting of LIN28A, FOXH1, LIN28B, GLIS1, p53-dominant negative, p53-P275S, L-MYC, NANOG, DPPA2, DPPA4, DPPA5, ZIC3, BCL-2, E-RAS, TPT1, SALL2, NAC1, DAX1, TERT, ZNF 206, FOXD3, REX1, UTF1, KLF2, KLF5, ESRRB, miR-291-3p, miR-294, miR-295, NR5A1, NR5A2, TBX3, MBD3sh, TH2A, TH2B, and P53DD.

A method for inducing differentiated cells from the pluripotent stem cell is not particularly limited. For example, a clamp may be produced from pluripotent stem cells according to the above-described first or second embodiment of the method for producing the clamp from the stem cells, and differentiated cells may be produced from the clamp according to the embodiment of the above-described method for producing the differentiated cells. The differentiated cell is, for example, a skin cell.

The induced differentiated cells are cultured in a culture medium to which a substance has been added. The substance is a substance that is capable of being added to a culture supernatant of autologous stem cells, a culture supernatant of heterologous stem cells, a beauty product, a cosmetic product, a hair tonic, or a hair growth promoter. Alternatively, the substance is a substance that is capable of being added to a pharmaceutical drug. Examples of the substance include a carrier, an excipient, a disintegrating agent, a buffering agent, an emulsifying agent, a suspending agent, a soothing agent, a stabilizer, a preservative, an antiseptic agent, and saline. Examples of the excipient include lactose, starch, sorbitol, D-mannitol, and white sugar. Examples of the disintegrating agent include carbonylmethyl cellulose and calcium carbonate. Examples of the buffering agent include a phosphate, a citrate, and an acetate. Examples of the emulsifying agent include gum arabic, sodium alginate, and tragacanth.

Examples of the suspending agent include glycerin monostearate, aluminum monostearate, methyl cellulose, carboxymethyl cellulose, hydroxymethyl cellulose, and sodium lauryl sulfate. Examples of the soothing agent include benzyl alcohol, chlorobutanol, and sorbitol. Examples of the stabilizer include propylene glycol and ascorbic acid. Examples of the preservative include phenol, benzalkonium chloride, benzyl alcohol, chlorobutanol, and methylparaben. Examples of the antiseptic agent include benzalkonium chloride, paraoxybenzoic acid, and chlorobutanol.

In addition, other examples of the substance include a culture medium in which stem cells have been cultured, a supernatant of a culture medium in which stem cells have been cultured, water, alcohol, surfactants (a cationic surfactant, an anionic surfactant, a nonionic surfactant, and amphoteric surfactant, and the like), moisturizing agents (glycerin, 1,3-butylene glycol, propylene glycol, propanediol, pentanediol, polyquaternium, an amino acid, urethane, a pyrrolidone carboxylate, nucleic acids, monosaccharides, and oligosaccharide, as well as derivative thereof and the like), thickeners (polysaccharides, a polyacrylic acid salt, a carboxyvinyl polymer, polyvinylpyrrolidone, polyvinyl alcohol, chitin, chitosan, alginic acid, carrageenan, xanthan gum, methyl cellulose, and the like, as well as derivative thereof and the like), wax, vaseline, a hydrocarbon-saturated fatty acid, an unsaturated fatty acid, silicon oil, as well as derivatives thereof and the like, triglycerides such as tri (capryl/capric acid) glyceryl) and glyceryl trioctanoate, ester oils such as isopropyl stearate, natural oil and fats (olive oil, camellia oil, avocado oil, almond oil, cacao oil, evening primrose oil, grape seed oil, macadamia nut oil, eucalyptus oil, rose hip oil, squalane, orange raffia, lanolin, and ceramide), antiseptic agents (an oxybenzoic acid derivative, a dehydroacetate, a photosensitizer, sorbic acid, phenoxyethanol, and the like, as well as derivative thereof and the like), bactericides (sulfur, trichlorocarbanilide, salicylic acid, zinc pyrithione, hinokitiol, and the like, as well as derivatives thereof and the like), ultraviolet absorbing agents (paraaminobenzoic acid, methoxycinnamic acid, and the like, as well as derivatives thereof and the like), antiinflammatory agents (allantoin, bisabolol, ε-aminocaproic acid, acetylfarnesyl cysteine, glycyrrhizic acid, and the like, as well as derivative thereof and the like), antioxidants (tocopherol, BHA, BHT, astaxanthin, and the like, as well as derivative thereof and the like), chelating agents (edetic acid, hydroxyethanediphosphonic acid, and the like, as well as derivative thereof and the like), animal and plant extracts (Angelica keiskei, aloe, rose fruit, scutellaria root, phellodendron bark, seaweed, Chinese quince, camomile, licorice, kiwi, cucumber, mulberry, white birch, Angelica acutiloba, garlic, Paeonia suffruticosa, hop, marronnier, lavender, lavender, rosemary, eucalyptus, milk, various peptides, placenta, royal jelly, a euglena extract, a hydrolyzed euglena extract, euglena oil, and the like, as well as purified products of components or fermented product thereof, and the like), pH adjusters (an inorganic acid, an inorganic acid salt, an organic acid, an organic acid, and the like, as well as derivative thereof and the like), vitamins (vitamin A and the like, vitamin B and the like, vitamin C, and vitamin D and the like, ubiquinone, nicotinic acid amide, and the like, as well as derivative thereof and the like), fermented liquids of yeast, aspergillus, and lactic bacteria, a Galactomyces culture solution, whitening agents (tranexamic acid, a tranexamic acid cetyl hydrochloride, 4-n-butylresorcinol, arbutin, kojic acid, ellagic acid, licorice flavonoid, niacinamide, vitamin C, and the like), ceramide/ceramide derivative, anti-wrinkle agents (retinol, retinal, and derivatives thereof, nicotinic acid amide, an oligopeptide, and the like, as well as derivatives thereof and the like, natural and synthetic components having a neutrophil elastase inhibiting action and MMP-1 and MMP-2 inhibiting action, and the like), titanium oxide, talc, mica, silica, zinc oxide, iron oxide, silicon, and powders obtained by processing these.

Other examples of the substance include a base (kaolin, bentonite, or the like), a gelling agent (a polyacrylic acid salt, polyvinyl alcohol, or the like), a sulfate, a hydrogen carbonate, a borate, and a pigment.

The culture medium for culturing the differentiated cells is not particularly limited; however, in a case where the differentiated cells are skin cells, the culture medium is, for example, DMEM to which FBS has been added.

It is determined whether or not a substance is administered or applied to a subject based on a response of the differentiated cells to the substance. For example, in a case where the differentiated cells have given a preferable response to the substance, it is determined that the substance is administered or applied to the subject. In a case where the differentiated cells have given an unpreferable response to the substance, it is determined that the substance is not administered or applied to the subject.

The response of the differentiated cells may be, for example, production of a product by the differentiated cells. In a case where the differentiated cell is a skin cell, examples of the product include collagen, hyaluronic acid, a fibrous blast growth factor (FGF) family, and a vascular endothelial cell growth factor (VEGF). In a case where the production amount of the product is increased by adding a substance to differentiated cells, it is determined that the differentiated cells have given a preferable response, and it is determined that the substance is applied or administered to a subject. In a case where the production amount of the product is reduced by adding a substance to differentiated cells, it is determined that the differentiated cells have given an unpreferable response, and it is determined that the substance is not applied or administered to a subject.

The response of the differentiated cells may be, for example, a survival rate of the differentiated cells. In a case where the survival rate of the differentiated cells is increased by adding a substance to the differentiated cells, it is determined that the differentiated cells have given a preferable response, and it is determined that the substance is applied or administered to a subject. In a case where the survival rate of the differentiated cells is decreased by adding a substance to the differentiated cells, it is determined that the differentiated cells have given an unpreferable response, and it is determined that the substance is not applied or administered to a subject.

The subject from which the somatic cells are provided and the subject to which a substance is applied or administered are the same person. The response of differentiated cells to a substance may vary from person to person. In contrast, it is possible to provide a substance suitable for the subject in a custom-made manner by observing a response of a substance in differentiated cells derived from somatic cells of the subject and providing the substance to which the differentiated cells give a preferred response, to the same subject.

### System for Screening Substance

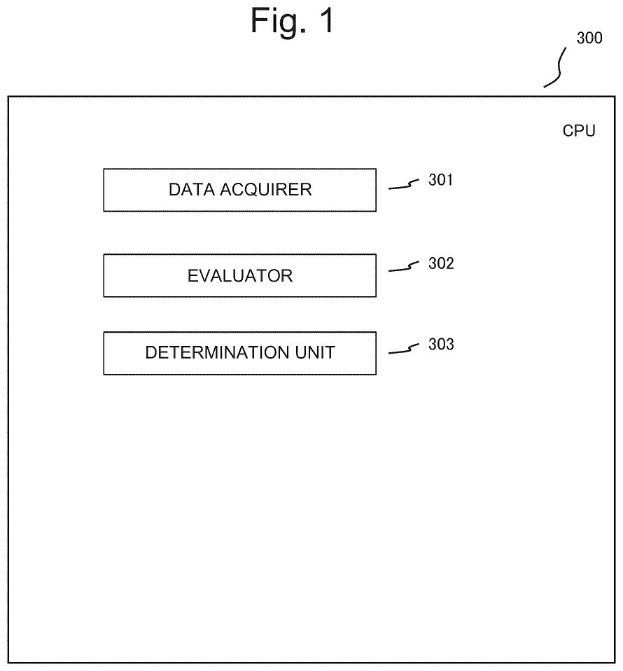

As illustrated in FIG. 1, an embodiment of a system for screening a substance includes a data acquirer 301 that is configured to acquire data on a response of differentiated cells in a case where a substance is added to the differentiated cells induced from pluripotent stem cells induced from somatic cells of a subject and the differentiated cells are cultured; an evaluator 302 that is configured to evaluate the substance based on the data on the response of the differentiated cells; and a determination unit 303 that is configured to determine whether or not to apply or administer the substance to the subject based on the evaluation. The data acquirer 301, the evaluator 302, and the determination unit 303 are included in, for example, a central processing unit (CPU) 300.

The data acquirer 301 acquires, for example, the data on the response of the differentiated cells to the substance, which has been described in the above-described method for screening a substance. For example, the data acquirer 301 may acquire the data on the response of the differentiated cells to each of a plurality of substances. In addition, the data acquirer 301 may acquire, as a control, the data on the response of the differentiated cells in a case where the substance has not been added to the differentiated cells.

The evaluator 302 evaluates whether or not the differentiated cells have given a preferable response to the substance. For example, as compared with the control, in a case where the production of the product is increased by the differentiated cells to which the substance has been added, the evaluator 302 evaluates that the differentiated cells have given a preferable response. In addition, as compared with the control, in a case where the production of the product is reduced by the differentiated cells to which the substance has been added, the evaluator 302 evaluates that the differentiated cells have given an unpreferable response.

In addition, for example, in a case where the survival rate of the differentiated cells to which the substance has been added is increased as compared with the control, the evaluator 302 evaluates that the differentiated cells have given a preferable response. In addition, in a case where the survival rate of the differentiated cells to which the substance has been added is reduced as compared with the control, the evaluator 302 evaluates that the differentiated cells have given an unpreferable response.

Alternatively, the evaluator 302 compares a plurality of substances and extracts a substance to which differentiated cells give a preferable response. For example, in a case where the survival rate of the differentiated cells to which a second substance among a plurality of substances has been added is increased as compared with a first substance, the evaluator 302 evaluates that the differentiated cells have given a preferable response to the second substance. In addition, the evaluator 302 evaluates that the differentiated cells have given an unpreferable response to the first substance.

In addition, for example, in a case where the survival rate of the differentiated cells to which a second substance among a plurality of substances has been added is increased as compared with a first substance, the evaluator 302 evaluates that the differentiated cells have given a preferable response to the second substance. In addition, the evaluator 302 evaluates that the differentiated cells have given an unpreferable response to the first substance.

The determination unit 303 determines to apply or administer a substance to a subject, the substance being a substance that has been evaluated by the evaluator 302 that the differentiated cells have given a preferable response, and it determines not to apply or administer a substance to a subject, the substance being a substance that has been evaluated that the differentiated cells have not given a preferable response. The determination unit 303 may transmit the determined item to an output device.

### Example 1

iPS cells were cultured in a feeder-free manner. The following culture medium was used: a culture medium (Puel, I Peace, Inc.) containing 80 ng/mL bFGF and 1% antibiotics (Gibco Antibiotic-Antimycotic (100×), registered trademark (Thermo Fisher Scientific, Inc.)) (hereinafter, referred to as “stem cell culture medium”). The same antibiotics are used in the following culture media. After the confluency reached 80% to 90%, TrypLE Select was added to the culture medium, iPS cells were left to stand at 37° C. for 5 to 10 minutes and detached from the culture vessel, and the stem cell culture medium containing the iPS cells were suspended with PIPETMAN to make the iPS cells single cells. A stem cell culture medium was added to the culture vessel to recover the single cells of the iPS cells, and the stem cell culture medium containing the iPS cells was centrifuged to recover the iPS cells.

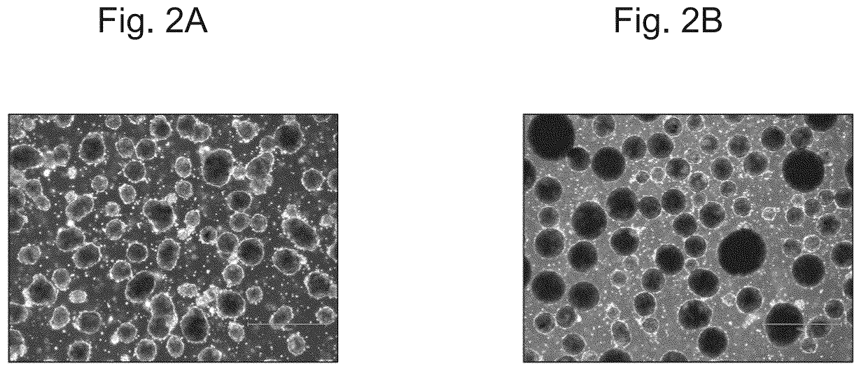

A Puel containing 1% antibiotics and containing no bFGF was prepared as an EB culture medium. The iPS cells were suspended in the EB culture medium to which a 0.1% ROCK inhibitor had been added to make the iPS cells single cells. 1×10⁶ cells of the iPS cells were suspended in 4 mL of the EB culture medium to which a 0.1% ROCK inhibitor and 1% Matrigel had been added, and the iPS cells were seeded as single cells in a PrimeSurface 3D culture 60 mm dish (hereinafter, referred to as a “first dish”) . After 2 days, 4 mL of the EB culture medium was added to the dish. FIG. 2A shows a photographic image of clamps on the 2nd day after seeding the iPS cells on the first dish. Then, half the amount of the EB culture medium in the first dish was replaced every two days until the clamps were formed. FIG. 2B shows a photographic image of clamps on the 6th day after seeding the iPS cells on the first dish.

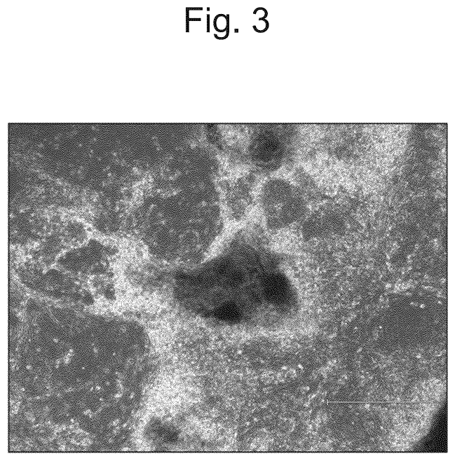

DMEM (high glucose) containing no ROCK inhibitor, to which 20% FBS, 64 μg/mL ascorbic acid, and a 1% antibiotics had been added, was prepared as a first fibroblast culture medium. In addition, a dish having an inner surface which had been coated with an extracellular substrate such as gelatin was prepared as a second dish. The EB culture medium and the clamps in the first dish were transferred to a tube, and the EB culture medium and the clamps were centrifuged to recover the clamps. The clamps were suspended in the first fibroblast culture medium, 10 or more clamps were seeded in the second dish per 9 cm², and the cells were cultured at 37° C. The first fibroblast culture medium in the second dish was replaced every two days. On the 8th day after the clamps were seeded on the second dish, it was observed that cells protruded from the clamps as shown in FIG. 3.

DMEM (high glucose) containing no ROCK inhibitor, to which 10% FBS and a 1% antibiotics had been added, was prepared as a second fibroblast culture medium. In addition, a dish having an inner surface which had not been coated with an extracellular substrate such as gelatin was prepared as a third dish. After the cells protruding outward from the clamps proliferated in the second dish, the cells were passaged according to the following procedure.

The first fibroblast culture medium was aspirated from the second dish, and PBS was put into the second dish to wash the cells with the PBS. PBS was aspirated from the second dish, 0.25% trypsin/EDTA was put into the second dish, and the second dish was left to stand at 37° C. for 3 minutes. After the cells were detached from the second dish, dissociation was carried out so that 50% or more of the cells were single cells. Then the first fibroblast culture medium was put into the second dish, and the first fibroblast culture medium in which the cells were suspended was aspirated from the second dish. The first fibroblast culture medium in which the cells were suspended was centrifuged to recover the cells. The recovered cells were suspended in the second fibroblast culture medium and seeded in the third dish at 1×10⁴ cells/cm to 5×10⁴ cells/cm.

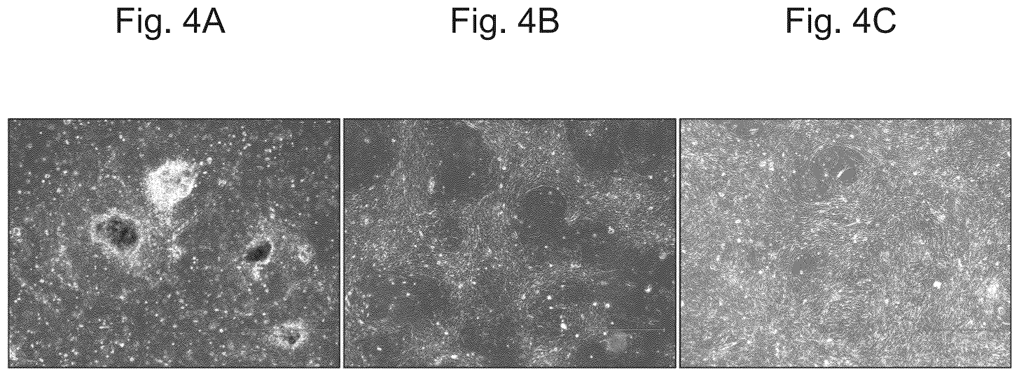

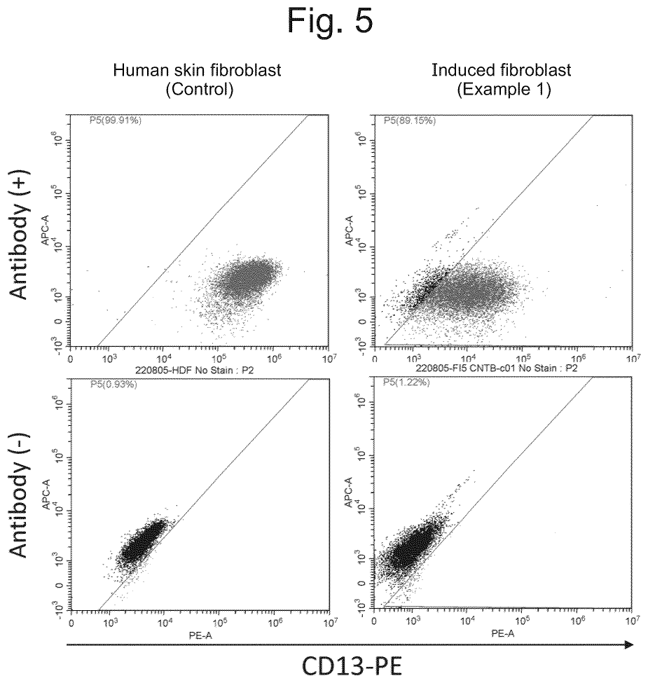

Then, the second fibroblast culture medium in the third dish was replaced every two days until the cells reached a confluency of 80% to 90%. The cells were passaged once every 4 to 7 days according to the same procedure as the procedure described above. FIG. 4A shows a photographic image of cells after the first passage, FIG. 4B shows a photographic image of cells after the third passage, and FIG. 4C shows a photographic image of the cells after the fourth passage. As the passage progressed, the morphology of the cells became the morphology of the fibroblast, and there were no TRA-1-60 positive cells and no epithelioid cells. As a result of analyzing the cells after the fourth passage by flow cytometry, CD13, which is a marker of the fibroblast, was positive as shown in FIG. 5.

### Comparative Example 1

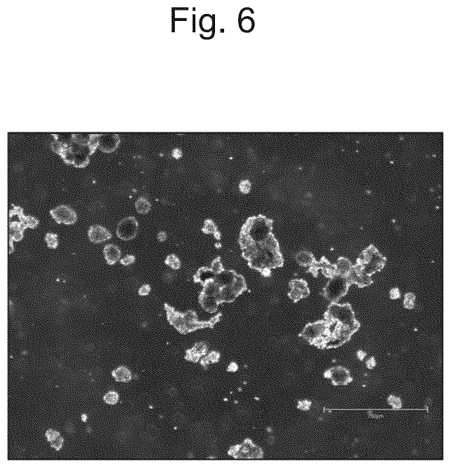

iPS cells were seeded as single cells on the first dish in the same manner as in Example 1, except that Matrigel was not added to the EB culture medium, and then the iPS cells were cultured. In this case, the clamp was barely formed as shown in FIG. 6.

### Example 2

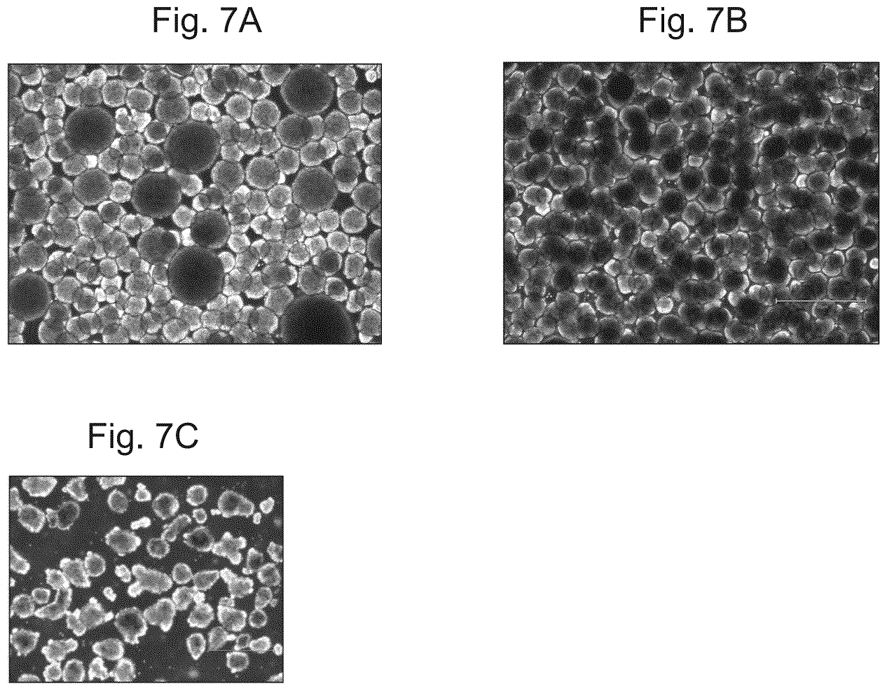

The same EB culture medium as in Example 1 was prepared. In addition, a 30 mL bioreactor (ABLE, REPROCELL Incorporated) was prepared. 20 mL of the EB culture medium to which a 0.1% ROCK inhibitor had been added was put into the bioreactor. 4×10⁶ cells of the iPS cells frozen in a freezing solution (STEM-CELLBANKER) were seeded in the EB culture medium in the bioreactor in a state of being suspended in the freezing solution without carrying out centrifugation to wash the freezing solution. The rotational speed was set to 16, and then cells were cultured at 37° C. In a case where the rotational speed was set to 16, a part of the iPS cells did not float, and at least 20% or more of the iPS cells came into contact with the bottom surface of the bioreactor. After 2 days, 10 mL of the EB culture medium was added into the bioreactor. Then, half the amount of the EB culture medium in the bioreactor was replaced every two days until the clamps were formed. FIG. 7A shows a photographic image of clamps on the 3rd day after seeding the iPS cells on the bioreactor, FIG. 7B shows a photographic image of clamps on the 4th day after seeding the iPS cells on the bioreactor, and FIG. 7C shows a photographic image of clamps on the 8th day after seeding the iPS cells on the bioreactor.

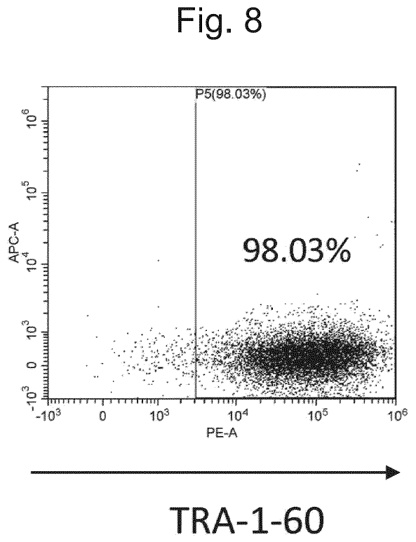

As a result of analyzing the cells on the 3rd day after seeding the iPS cells in the bioreactor by flow cytometry, TRA1-60, which is a marker of the pluripotent stem cell, was positive as shown in FIG. 8. In addition, on the 11th day after the iPS cells were seeded in the bioreactor, RNA was extracted from the clamps, and the expression levels of PAX6, Brachyury, and SOX17 were analyzed by quantitative PCR. Undifferentiated iPS cells were used as a control. PAX6 is a marker of the ectoderm (nerve system). Brachyury is a marker of the mesoderm. SOX17 is a marker of the endotherm (lung, liver, pancreas, muscle, and a digestive organ such as stomach or intestine).

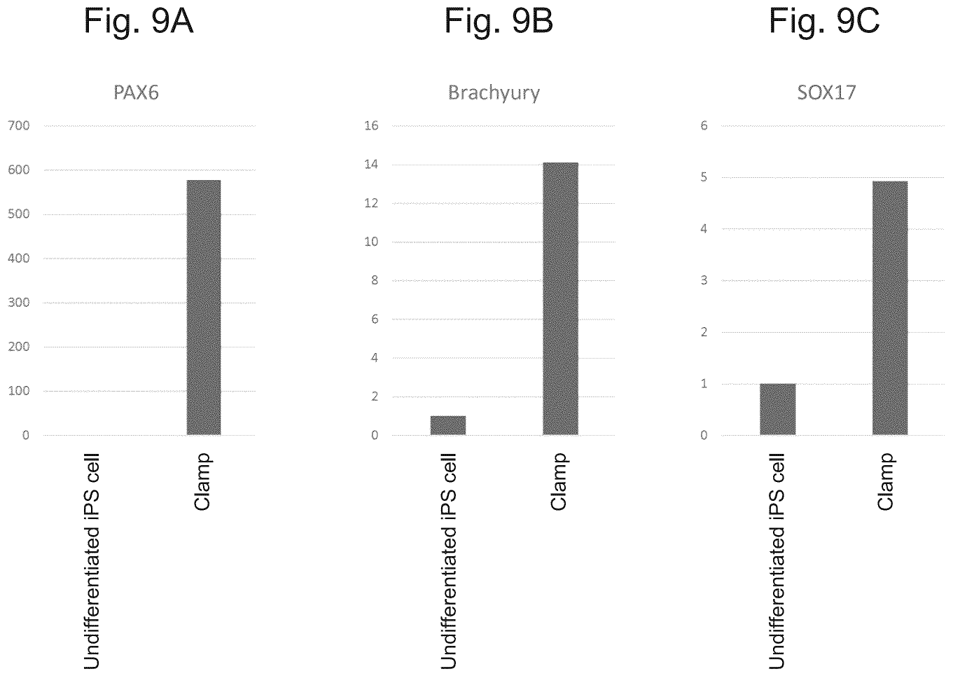

FIG. 9A, FIG. 9B, and FIG. 9C respectively show the expression levels of PAX6, Brachyury, and SOX17 in the clamp in a case where the expression levels of PAX6, Brachyury, and SOX17 in the undifferentiated iPS cells are normalized to 1. In the clamp, PAX6, Brachyury, and SOX17 were positive. As a result, it was shown that, in the bioreactor according to the present example, the three germ layers are capable of being induced from iPS cells. It is noted that in a case where the rotational speed of the bioreactor was increased after the clamps were formed so that less than 20% of the clamps did not come into contact with the bottom surface of the bioreactor, the sizes of the plurality of clamps become uniform, and the efficiency of differentiation into the three germ layers increased as compared with a case where the rotational speed of the bioreactor was not increased.

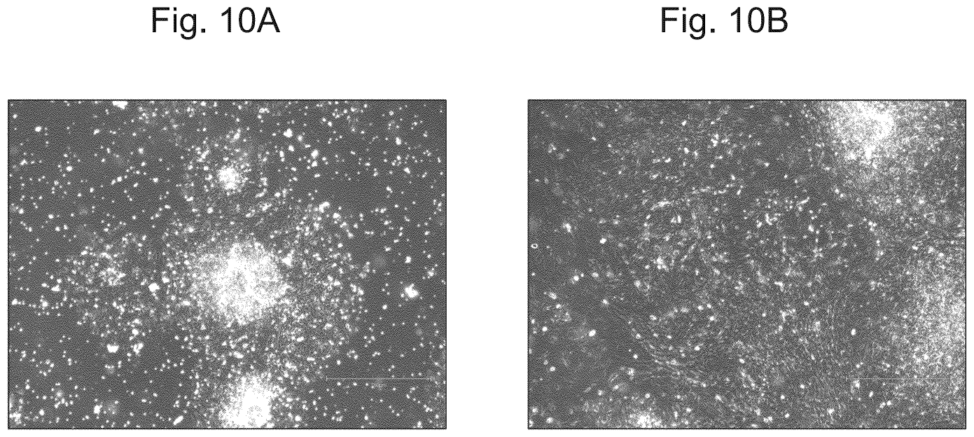

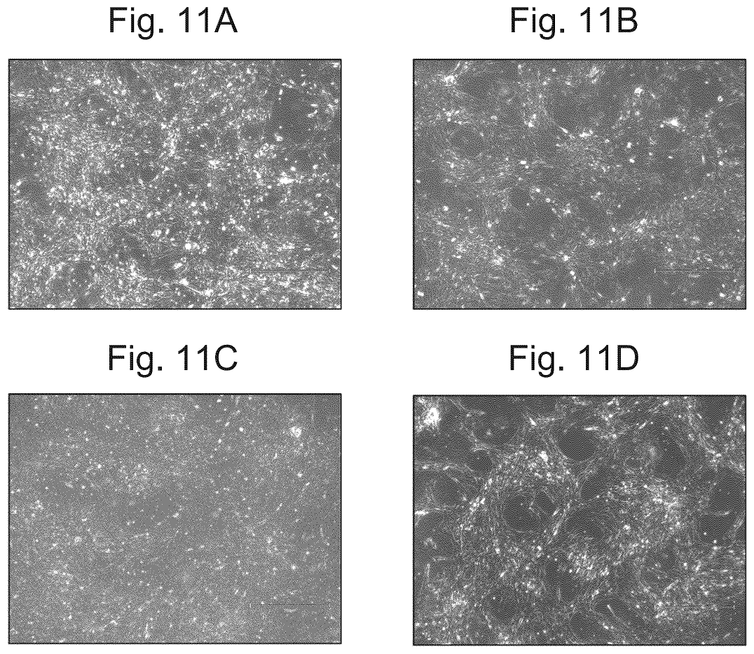

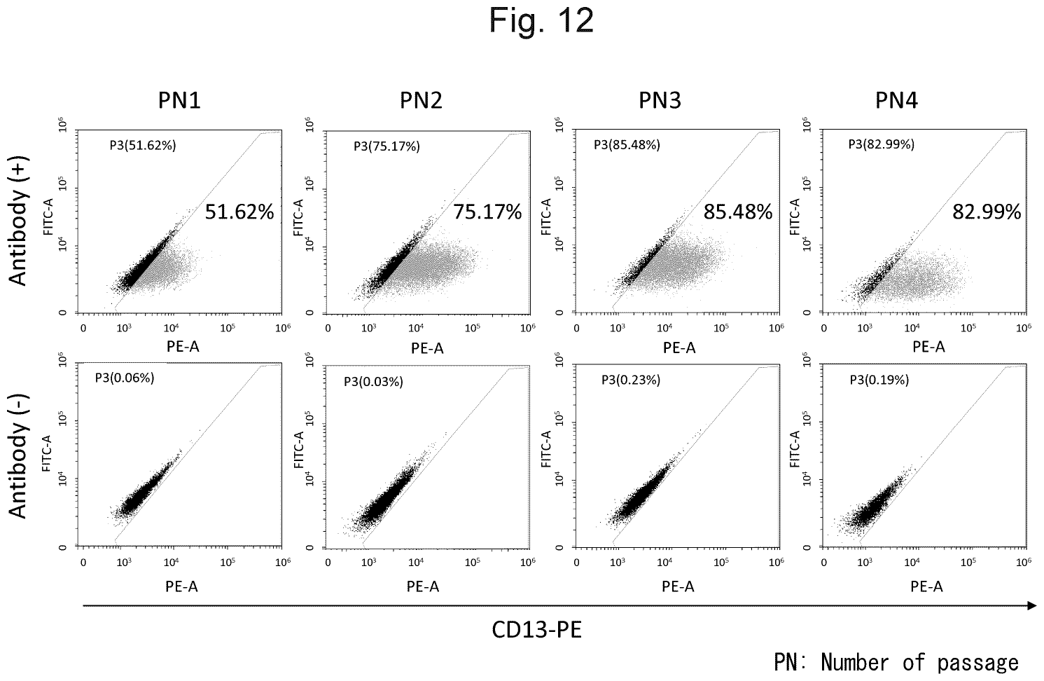

After such clamps as shown in FIG. 7C were formed, the clamps were seeded in a dish having an inner surface coated with gelatin, in the same manner as in Example 1, and the cells were subcultured. FIG. 10A shows a photographic image of the cells on the 2nd day after the clamps were seeded in a dish having an inner surface coated with gelatin, and FIG. 10B shows a photographic image of the cells on the 8th day. In addition, FIG. 11A shows a photographic image of cells after the first passage, FIG. 11B shows a photographic image of cells after the second passage, FIG. 11C shows a photographic image of cells after the third passage, and FIG. 11D shows a photographic image of cells after the fourth passage. As the passage progressed, the morphology of the cells became the morphology of the fibroblast. In addition, as a result of analyzing the cells after each of the first to fourth passages by flow cytometry, the positive rate for CD13, which is a fibroblast marker, increased as shown in FIG. 12.

### Comparative Example 2

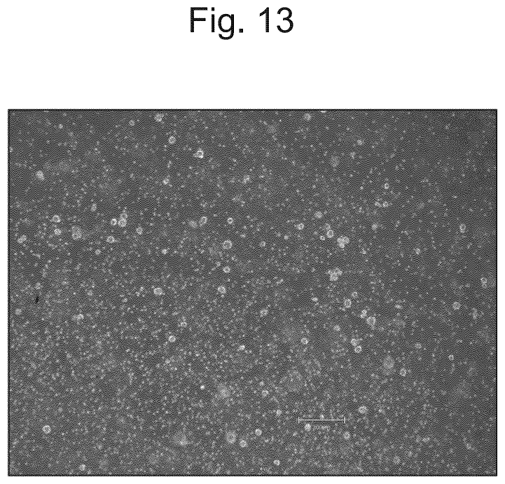

iPS cells were cultured in the same manner as in Example 2, except that the rotational speed of the stirring device of the bioreactor was increased, the iPS cells were allowed to float in the EB culture medium, and at least less than 20% of the iPS cells allowed to come into contact with the bottom surface of the bioreactor. In this case, the clamp was barely formed as shown in FIG. 13.

### Example 3

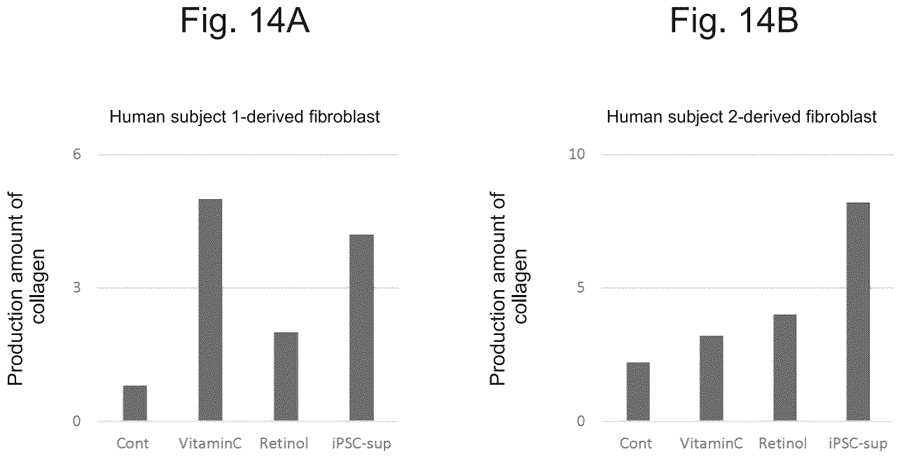

Skin cells were collected from a human subject 1 and a human subject 2, and iPS cells were induced from the skin cells from the respective subjects. Further, in the same manner as in Example 2, fibroblasts were induced from the human subject 1-derived iPS cells, and fibroblasts were induced from the human subject 2-derived iPS cells. As a result of adding each of vitamin C, retinol, and the supernatant of the iPS cell culture medium to the human subject 1-derived induced fibroblasts, the amount of the collagen produced was increased as shown in FIG. 14A, as compared with the control in which the addition was not carried out. In addition, as a result of adding each of vitamin C, retinol, and the supernatant of the iPS cell culture medium to the human subject 2-derived induced fibroblasts, the amount of the collagen produced was increased as shown in FIG. 14B, as compared with the control in which the addition was not carried out. However, in a case where FIG. 14A and FIG. 14B were compared, in the induced fibroblasts derived from the human target 1, vitamin C was most effective in increasing the production amount of collagen; however, in the induced fibroblasts derived from the human target 2, the iPS cell culture medium was most effective in increasing the production amount of collagen. This result indicates that it is preferable to add vitamin C to a cosmetic product that is provided to the human subject 1, and it is preferable to add the iPS cell culture medium to a cosmetic product that is provided to the human subject 2.

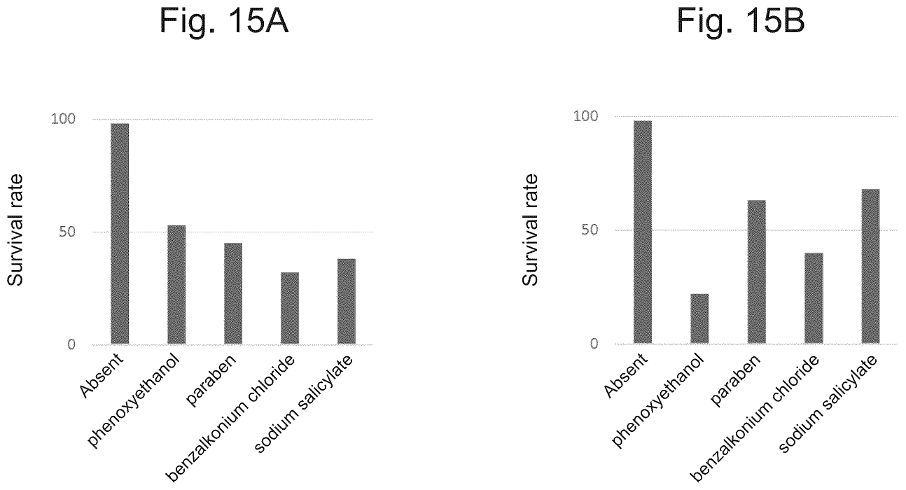

In addition, phenoxyethanol, paraben, benzalkonium chloride, and sodium salicylate, which are respectively antiseptic agents, were prepared. As a result of adding each of phenoxyethanol, paraben, benzalkonium chloride, and sodium salicylate to the human subject 1-derived induced fibroblasts, the survival rate of cells to which phenoxyethanol had been added was highest as shown in FIG. 15A. As a result of adding each of phenoxyethanol, paraben, benzalkonium chloride, and sodium salicylate to human subject 2-derived induced fibroblasts, the survival rate of cells to which sodium salicylate had been added was highest as shown in FIG. 15B. This result indicates that it is preferable to add phenoxyethanol to a cosmetic product that is provided to the human subject 1, and it is preferable to add sodium salicylate to a cosmetic product that is provided to the human subject 2.

### Reference Table

300 CPU
301 data acquirer
302 evaluator
303 determination unit

1. A method for screening a substance, comprising:
inducing pluripotent stem cells from somatic cells of a subject;
inducing differentiated cells from the pluripotent stem cells;
adding a substance to the differentiated cells and culturing the differentiated cells; and
determining whether or not to administer or apply the substance to the subject based on a response of the differentiated cells to the substance.

2. The method according to claim 1, wherein the subject from which the somatic cells are provided and the subject to which the substance is administered or applied are the same person.

3. The method according to claim 1, wherein the substance is an additive of a beauty product, a cosmetic product, a hair tonic, or a hair growth promoter.

4. The method according to claim 1, wherein the substance is an additive of a pharmaceutical drug.

5. The method according to claim 1, wherein the substance is a vitamin.

6. The method according to claim 1, wherein the substance is an antiseptic agent.

7. The method according to claim 1, wherein the response of the differentiated cells is a survival rate of the differentiated cells.

8. A method for administering or applying a substance, comprising:
inducing pluripotent stem cells from somatic cells of a subject;
inducing differentiated cells from the pluripotent stem cells;
adding a substance to the differentiated cells and culturing the differentiated cells;
determining whether or not to administer or apply the substance to the subject based on a response of the differentiated cells to the substance; and
administering or applying the substance to the subject, where the substance has been determined to be administered or applied.

9. The method according to claim 8, wherein the subject from which the somatic cells are provided and the subject to which the substance is administered or applied are the same person.

10. The method according to claim 8, wherein the substance is an additive of a beauty product, a cosmetic product, a hair tonic, or a hair growth promoter.

11. The method according to claim 8, wherein the substance is an additive of a pharmaceutical drug.

12. The method according to claim 8, wherein the substance is a vitamin.

13. The method according to claim 8, wherein the substance is an antiseptic agent.

14. The method according to claim 8, wherein the response of the differentiated cells is a survival rate of the differentiated cells.

15. A system for screening a substance, comprising:
a data acquirer configured to acquire data on a response of differentiated cells in a case where a substance is added to the differentiated cells induced from pluripotent stem cells induced from somatic cells of a subject and the differentiated cells are cultured;
an evaluator configured to evaluate the substance based on the data on the response of the differentiated cells; and
a determination unit configured to determine whether or not to administer or apply the substance to the subject based on the evaluation.

16. The system according to claim 15, wherein the subject from which the somatic cells are provided and the subject to which the substance is administered or applied are the same person.

17. The system according to claim 15, wherein the substance is an additive of a beauty product, a cosmetic product, a hair tonic, or a hair growth promoter.

18. The system according to claim 15, wherein the substance is an additive of a pharmaceutical drug.

19. The system according to claim 15, wherein the substance is an antiseptic agent.

20. The system according to claim 15, wherein the response of the differentiated cells is a survival rate of the differentiated cells.
